(19) United States
(12) Patent Application Publication
(10) Pub. No.: US 2011/0236890 A1
(43) Pub. Date: Sep. 29, 2011

(54) METHOD OF SCREENING FOR CANCER BY DETECTING MUTATIONS IN THE DELTA-CATENIN GENE PROMOTER AND 5'-UNTRANSLATED REGION

(76) Inventors: Qun Lu, Greenville, NC (US); Tao Wang, Namjing (CN); Yan-Hua Chen, Greenville, NC (US)

(21) Appl. No.: 12/738,665

(22) PCT Filed: Nov. 19, 2008
(86) PCT No.: PCT/US08/12906

§ 371 Date: Sep. 7, 2010

Related U.S. Application Data
(60) Provisional application No. 60/989,158, filed on Nov. 20, 2007.

(51) Int. Cl. C12Q 1/68

(52) U.S. Cl. 435/6.11; 435/6.12; 435/6.14

(57) ABSTRACT

A method for screening for risk of cancer in a subject is carried out by detecting the presence or absence of at least one mutation in the delta-catenin gene promoter or 5′ untranslated region in a biological sample from said subject, the presence of such mutation or an increased frequency of mutation indicating said subject is afflicted with or at least at risk of developing cancer.

### GOVERNMENT SUPPORT

This invention was made with government support under grant numbers CA111891 from the National Institutes of Health and PC040569 from the Department of Defense. The US Government has certain rights to this invention.

### BACKGROUND OF THE INVENTION

Prostate cancer is a major cause of death among men in Western countries. The current protocol for detection of this cancer involves testing for prostate-specific antigen (PSA) levels. If PSA levels are found to be high (4 ng/ml), a tissue biopsy is performed. Unfortunately, PSA testing is limited by the fact that it lacks sensitivity and it does not distinguish between prostate cancer and benign prostate hyperplasia. As a result, many men either are not identified as having the disease or because of false positive tests are subjected to the invasive tissue biopsies when they do not have the disease. A much more specific and less invasive diagnostic test is needed for early detection of this disease.

Delta-catenin presents itself as an improved alternative to the PSA/biopsy tests currently utilized for prostate cancer detection. Delta-catenin was first identified and patented (U.S. Pat. No. 6,258,929) as a neurospecific protein, alternatively named ALARM. At the time, the protein was believed to be expressed almost exclusively in brain tissue. However, Burger, et al. (Int. J. Cancer 100, 228-237 (2002)) subsequently found the messenger RNA for delta-catenin to be expressed in prostate cancer tumors with the delta-catenin transcripts being localized to the glandular secretory cells. Unlike PSA, delta-catenin was capable of distinguishing between prostate cancer and benign prostate hyperplasia. Burger et al. noted a possible diagnostic role for delta-catenin in prostate cancer detection. However, they also pointed out that a significant difficulty remained in development of this tool since delta-catenin had only been detected in glandular secretory epithelial cells in prostate tissues and had not been found in prostate stroma or bodily fluids, such as serum or urine.

### SUMMARY OF THE INVENTION

A first aspect of the present invention is a method for screening for risk of cancer in a subject comprising the steps of: detecting the presence or absence of at least one mutation in the delta-catenin gene promoter or 5′ untranslated region in a biological sample from said subject; the presence of said mutation or an increased frequency of mutation (e.g., as compared to an unafflicted subject or a population or group of unafflicted subjects) indicating said subject is afflicted with or at least at risk of developing cancer.

It will be appreciated that the instant invention concerns, among other things, detecting increased incidences of nucleotide polymorphic changes in the delta-catenin gene promoter or 5′ untranslated region in a biological sample from a subject, and determining or indicating an increased risk of or affliction with cancer from the increased incidence of such changes detected (as compared to subjects not afflicted with that cancer or not at increased risk).

Mutations that may be detected include, but are not limited to, delta-catenenin gene promoter or 5′ untranslated region mutations set forth in Table 1 below. In some embodiments, the mutation is not −9 G-A (when the translation initiation codon site is treated as +1) (or “G137A” when the transcription start nucleotide treated as +1), or the −9 G-A mutation is excluded therefrom, or at least one additional mutation from Table 1 additional to −9G-A is detected.

Subjects may be male or female, and cancers to be screened include but are not limited to lung, breast, colon, prostate, esophageal, ovarian, pancreatic, adrenal, skin cancer and leukemia. In some embodiments the subject is male, and said cancer is prostate cancer.

A particular embodiment of the invention is, in a method for screening for cancer in a subject by detecting the presence of a cadherin, prostate specific antigen, and/or p120 cancer biomarker in said subject, the improvement comprising the steps of: detecting the presence or absence of at least one mutation in the delta-catenin gene promoter or 5′ untranslated region in a biological sample from said subject; the presence of said mutation or an increased frequency of mutation indicating said subject is afflicted with or at least at risk of developing cancer. Subjects, mutations, and cancers may be as described above.

A further aspect of the invention is the use of a means of detecting the presence or absence of mutation in the delta-catenin gene promoter or 5′ untranslated region in a biological sample from a subject for carrying out a method as described above.

A further aspect of the invention is a kit comprising a means of detecting the presence or absence of mutation in the delta-catenin gene promoter or 5′ untranslated region in a biological sample from a subject for carrying out a method as described above, the kit optionally including instructions for carrying out a method as described above.

The foregoing and other objects and aspects of the present invention are explained in greater detail in the drawings herein and the specification set forth below.

### DETAILED DESCRIPTION OF THE PREFERRED EMBODIMENTS

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which does not depart from the instant invention. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

“Cancer” as used here includes but is not limited to brain, lung, breast, colon, prostate, esophageal, ovarian, pancreatic, adrenal, skin cancer and leukemia cells.

“Subject” as used herein refers to animal subjects, particularly mammalian subjects, including but not limited to, humans, non-human primates, dogs, cats, rabbits, goats, horses, pigs, and cattle. The subject may be a male subject or a female subject and may be of all ages including infant, juvenile, adolescent and adult subject.

“Fluid sample” or “body fluid sample” as used herein includes but is not limited to blood samples, seminal fluid, urine, and fine needle aspirates from suspected afflicted organs such as the prostate in a subject.

“Blood sample” as used here refers to whole blood, blood plasma, blood serum or any fraction thereof, so long as the fraction contains (in subject with cancer) delta-catenin as described herein.

The disclosures of all US Patent references cited herein are to be incorporated by reference herein in their entirety.

The present invention is, as noted above, drawn to methods for detection of cancer utilizing delta-catenin. Detection may be for diagnostic or prognostic purposes, may be for general screening purposes, may be for targeting cancer in chemotherapy, or may be for the purpose of determining if a subject is at risk of developing cancer, confirm a diagnosis, indicate the reoccurrence of cancer, etc.

As noted above, the subject may be a human subject, or an animal subject for veterinary Gor drug screening or development purposes, with examples of suitable animal subjects including but not limited to dogs, cats, rabbits, goats, horses, pigs, cattle, etc. The subjects may be a male subject or a female subject; the subject may be of any age including infant, juvenile, adolescent and adult subjects.

The present invention may be used to detect any type of cancer, including but not limited to esophageal, lung, breast, colon, ovarian, pancreatic, adrenal, skin cancer or leukemia. In one preferred embodiment of the invention, the cancer to be detected is prostate cancer.

Amplification of nucleic acids may be carried out by any suitable technique, including but not limited to polymerase chain reaction (including, for RNA amplification, reverse-transcriptase polymerase chain reaction), ligase chain reaction, strand displacement amplification, transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Q.beta. replicase system (see P. Lizardi et al., Biotechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). The bases incorporated into the amplification product may be natural or modified bases (modified before or after amplification), and the bases may be selected to optimize subsequent electrochemical detection steps. Techniques for amplification are known and described in, among other things, U.S. Pat. Nos. 4,683,195; 4,683,202; 4,800,159; and 4,965,188; G. Walker et al., Proc. Natl. Acad. Sci. USA 89, 392-396 (1992); G. Walker et al., Nucleic Acids Res. 20, 1691-1696 (1992); R. Weiss, Science 254, 1292 (1991).

A biological sample may be a cell sample, with an intervening culturing step being performed between the time the cell sample is collected from the subject and the immunoassay is carried out on the biological sample. In some embodiments a sample is obtained by using rectal massage to release some cells into the urine and then collect the urine to perform PCR; in some embodiments void urine is collected and cell debris spun down therein to perform PCR; in some embodiments needle biopsy is carried out to obtain a tissue sample on which PCR is performed.

A biological sample may also be a cell, cell debris, or stroma sample, with an intervening step being performed before the sample is collected from the subject to enhance the sensitivities of the assay.

In general, the step of detecting the polymorphism of interest may be carried out by collecting a biological sample containing DNA from the subject, and then determining the presence or absence of DNA containing the polymorphism of interest in the biological sample. Any biological sample which contains the DNA of that subject may be employed, including tissue samples and blood samples, with blood cells being a particularly convenient source. The nucleotide sequence of the human delta-catenin gene promoter is known and suitable probes, restriction enzyme digestion techniques, or other means of detecting the polymorphism may be implemented based on this known sequence in accordance with standard techniques. See, e.g., U.S. Pat. Nos. 6,027,896 and 5,767,248 to A. Roses et al. (Applicants specifically intend that the disclosures of all United States patent references cited herein be incorporated by reference herein in their entirety).

In describing the mutations or polymorphisms disclosed herein the naming method is as follows: nucleotide position in promoter] [replaced nucleotide]−[alternate nucleotide].

The polymorphisms described herein can be detected in accordance with known techniques based upon the known sequence information of the promoter and the information provided herein. Novel nucleic acid sequences and proteins described herein can be isolated from human sources based upon the information provided herein or produced by other means such as site-directed mutagenesis of known or available nucleic acids, coupled as necessary with techniques for the production of recombinant proteins known in the art.

Determining the presence or absence of DNA containing a polymorphism or mutation of interest may be carried out with an oligonucleotide probe labeled with a suitable detectable group, or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the polymorphism of interest. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. Patent references cited herein be incorporated herein by reference).

Amplification of a selected, or target, nucleic acid sequence may be carried out by any suitable means. See generally D. Kwoh and T. Kwoh, Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad. Sci. USA 87, 1874-1878 (1990)), the QB replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra).

DNA amplification techniques such as the foregoing can involve the use of a probe, a pair of probes, or two pairs of probes which specifically bind to DNA containing the polymorphism of interest, but do not bind to DNA that does not contain the polymorphism of interest under the same hybridization conditions, and which serve as the primer or primers for the amplification of the DNA or a portion thereof in the amplification reaction. Such probes are sometimes referred to as amplification probes or primers herein.

In general, an oligonucleotide probe which is used to detect DNA containing a polymorphism or mutation of interest is an oligonucleotide probe which binds to DNA encoding that mutation or polymorphism, but does not bind to DNA that does not contain the mutation or polymorphism under the same hybridization conditions. The oligonucleotide probe is labeled with a suitable detectable group, such as those set forth below in connection with antibodies. Such probes are sometimes referred to as detection probes or primers herein.

Probes and primers, including those for either amplification and/or protection, are nucleotides (including naturally occurring nucleotides such as DNA and synthetic and/or modified nucleotides) are any suitable length, but are typically from 5, 6, or 8 nucleotides in length up to 40, 50 or 60 nucleotides in length, or more. Such probes and or primers may be immobilized on or coupled to a solid support such as a bead, chip, pin, or microtiter plate well in accordance with known techniques, and/or coupled to or labeled with a detectable group such as a fluorescent compound, a chemiluminescent compound, a radioactive element, or an enzyme in accordance with known techniques.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all ApoE allelic types, the types can be distinguished by hybridization with allelic specific probe, by restriction endonuclease digestion, by electrophoresis on denaturing gradient gels, or other techniques.

Ligase chain reaction (LCR) is also carried out in accordance with known techniques. See, e.g., R. Weiss, Science 254, 1292 (1991). In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

It will be readily appreciated that the detecting steps described herein may be carried out directly or indirectly. For example, a polymorphism or mutation could be detected by measuring by digestion with restriction enzymes, detection of markers that are linked to the mutation or polymorphism, etc.

Kits useful for carrying out the methods of the present invention will, in general, comprise one or more oligonucleotide probes and other reagents for carrying out the methods as described above, such as restriction enzymes, optionally packaged with suitable instructions for carrying out the methods.

The new polymorphisms described herein provide novel nucleic acids encoding the human promoter, along with probes such as described above that bind selectively thereto.

Another aspect of the invention involves the use of a combination of biomarkers to reduce the frequency of false positives or false negatives by the use of any one biomarker alone. For example, where a subject is tested for b-catenin in the manner described herein, that subject may also be tested for the presence of another biomarker. For example, the presence of at least two biomarkers indicates that the subject is more likely to be afflicted with cancer than if only one biomarker is found in that subject; the absence of at least two biomarkers indicates the subject is more likely to be free of cancer than if only one biomarker is found in that subject; the presence of one biomarker in a subject indicates the subject is more likely to be afflicted with cancer than if the subject is found to be free of another, different, biomarker, etc. Particular biomarkers that may be used in combination with the methods of testing for δ-catenin as described herein include tight junction and adherens junction proteins such as claudin and cadherin (particularly E-cadherin), prostate specific antigen, and p120 (particularly p120c^{cth}).

The presence or absence of other cancer biomarkers may be detected in accordance with known techniques. Methods of detecting, diagnosing or screening for cancer (particularly prostate cancer) by detecting the presence of prostate specific antigen (PSA) are known and described in, among other things, U.S. Pat. Nos. 5,614,372; 5,840,501; 6,300,088; 6,361,955; 6,383,759; 6,423,503; and 6,482,599. Methods of detecting, diagnosing or screening for cancer (including prostate cancer) by detecting cadherins such as E-, OB-, N- and T-cadherin (particularly E-cadherin) are known and described in, among other things, U.S. Pat. Nos. 5,597,725; 5,811,518; 5,997,866; 6,682,901; and 6,723,320. Methods of detecting, diagnosing or screening for cancer by detecting p120 (including p120^{ctn}) are known and described in, among other things, U.S. Pat. No. 4,902,615. The disclosures of all patent references cited herein are to be incorporated by reference herein in their entirety.

The present invention is illustrated in greater detail in the following non-limiting Examples.

### EXPERIMENTAL

### Delta-Catenin Gene (CTNND2)

### Mutations or Polymorphisms in Prostate Cancer

δ-Catenin (NPRAP/Neurojungin) is a unique β-catenin/armadillo domain-containing p120^{ctn} subfamily protein in that it is primarily expressed in the central nervous system. However, δ-catenin is upregulated in over 80% of human prostatic adenocarcinomas although how its expression is regulated is currently unclear. Analyses of δ-catenin CpG islands in the promoter region in benign and prostate cancer specimens revealed no significant hyper- or hypomethylation. Real time PCR analyses found no evidence of gene amplification in δ-catenin. However, we have observed an increased incidence of mutations in the promoter and 5′-untranslated region of δ-catenin gene when compared to that of benign prostatic tissue specimens as well as to that of peripheral blood samples of normal control subjects. Mutations are identified in TABLE 1 below. The δ-Catenin promoter region contains strong consensus binding domains for transcription factors such as E2F, Pax6 and LEF-1. Ectopic expression of E2F, but not LEF-1 or Notch intracellular domain (NICD), significantly increased δ-catenin promoter activity. This activation can be suppressed by the co-expression of Rb, an E2F inactivating protein. In addition, overexpression of E2F in CWR22Rv-1 prostate cancer cells increased δ-catenin expression. These studies indicate that δ-catenin promoter as a target for upregulation in human prostate cancer.

In Table 1, the translation initiation codon is treated as +1 (for example, G137A when the transcription start site is treated as +1 is −9G to A, or −9 G-A, when the translation initiation codon is treated as +1).

TABLE 1

−807 T-C, −710 T-A, −655 A-C, −449 C-T, −331 C-T, −235 G-A,
−172 C-T, −101 G-C, −99 A-G, −98 G-C, −98 G-T, −97 A-C,
−97 Insert C, −96 T-A, −94 C-T, −93 T-A, −91 C-G, −90 A-G,
−81 A-C, −79 G-A, −76 A-T, −72 C-T, −70 T-C, −60 G-A,
−58 C-T, −57 G-C, −55 G-C, −54 C-T, −53 G-C, −51 C-T,
−50 G-A, −49 C-T, −47 G-A, −45 G-A, −42 C-T, −33 C-T,
−17 T-A, −13 C-T, −10 C-T, and −9 G-A.
Notes on Table 1:
1. −9 G-A: True mutation and functional*;
2. −13 C-T: True mutation with repeated occurrence but function unknown**;
3. −58 C-T: Potential polymorphism with unknown function***;
4. −98 G-C/T and also −97A-C or insert C: Repeated occurrence with unknown function#;
5. −17 T-A: True mutation with unknown function##
*this indicates that mutation occurred in cancerous but not adjacent benign glands, also the mutant shows capability to increase delta-catenin expression;
**this indicates that mutation occurred from more than one patients in cancerous but not adjacent benign glands, but the capability to increase delta-catenin expression is unknown;
***this indicates that mutation occurred in both cancerous and adjacent benign glands;
#this indicates that mutation was seen from more than one patient;
##this indicates that mutation occurred in cancerous but not adjacent benign glands, although the capability to increase delta-catenin expression is unknown;

The foregoing is illustrative of the present invention, and is not to be construed as limiting thereof. The invention is defined by the following claims with equivalents of the claims to be included therein.

1. A method for screening for risk of cancer in a subject comprising the steps of:
detecting the presence or absence of at least one mutation in the delta-catenin gene promoter or 5′ untranslated region in a biological sample from said subject;
the presence of said mutation or an increased frequency of mutation indicating said subject is afflicted with or at least at risk of developing cancer.

2. The method of claim 1, wherein said at least one mutation is selected from the group consisting of delta-catenenin gene promoter or 5′ untranslated region mutations −807 T-C, −710 T-A, −655 A-C, −449 C-T, −331 C-T, −235 G-A, −172 C-T, −101 G-C, −99 A-G, −98 G-C, −98 G-T, −97 A-C, −97 Insert C, −96 T-A, −94 C-T, −93 T-A, −91 C-G, −90 A-G, −81 A-C, −79 G-A, −76 A-T, −72 C-T, −70 T-C, −60 G-A, −58 C-T, −57 G-C, −55 G-C, −54 C-T, −53 G-C, −51 C-T, −50 G-A, −49 C-T, −47 G-A, −45 G-A, −42 C-T, −33 C-T, −17 T-A, −13 C-T, −10 C-T, and −9 G-A (with the translation initiation codon site treated as +1).

3. The method of claim 1, wherein said mutation is not −9 G-A (when the translation initiation codon site is treated as +1) (or “G137A” when the transcription start nucleotide treated as +1).

4. The method of claim 1, wherein said mutation is −9 G-A.

5. The method of claim 1 wherein said subject is a human.

6. The method of claim 1 wherein said subject is male.

7. The method of claim 1 wherein said subject is female.

8. The method of claim 1 wherein said cancer is lung, breast, colon, prostate, esophageal, ovarian, pancreatic, adrenal, skin cancer or leukemia.

9. The method of claim 1, wherein said subject is male, and said cancer is prostate cancer.

10. In a method for screening for cancer in a subject by detecting the presence of a cadherin, prostate specific antigen, and/or p120 cancer biomarker in said subject, the improvement comprising the steps of:
detecting the presence or absence of at least one mutation in the delta-catenin gene promoter or 5′ untranslated region in a biological sample from said subject; the presence of said mutation or an increased frequency of mutation indicating said subject is afflicted with or at least at risk of developing cancer.

11. The method of claim 10, wherein said at least one mutation is selected from the group consisting of delta-catenenin gene promoter or 5′ untranslated region mutations −807 T-C, −710 T-A, −655 A-C, −449 C-T, −331 C-T, −235 G-A, −172 C-T, −101 G-C, −99 A-G, −98 G-C, −98 G-T, −97 A-C, −97 Insert C, −96 T-A, −94 C-T, −93 T-A, −91 C-G, −90 A-G, −81 A-C, −79 G-A, −76 A-T, −72 C-T, −70 T-C, −60 G-A, −58 C-T, −57 G-C, −55 G-C, −54 C-T, −53 G-C, −51 C-T, −50 G-A, −49 C-T, −47 G-A, −45 G-A, −42 C-T, −33 C-T, −17 T-A, −13 C-T, −10 C-T, and −9 G-A (with the translation initiation codon site is treated as +1).

12. The method of claim 11, wherein said mutation is not −9 G-A (when the translation initiation codon site is treated as +1; or “G137A” when the transcription start nucleotide is treated as +1).

13. The method of claim 11, wherein said mutation is −9 G-A.

14. The method of claim 10 wherein said subject is a human.

15. The method of claim 10 wherein said subject is male.

16. The method of claim 10 wherein said subject is female.

17. The method of claim 10 wherein said cancer is lung, breast, colon, prostate, esophageal, ovarian, pancreatic, adrenal, skin cancer or leukemia.

18. The method of claim 10, wherein said subject is male and said cancer is prostate cancer.

19. The method of claim 1, wherein said detecting step comprises: (i) amplifying nucleic acid of said subject in said biological sample to produce an amplification product, and then (ii) detecting the presence or absence of at least one mutation in said amplification product (ii).

20. The method of claim 1, wherein said detecting step is carried out by polymerase chain reaction (PCR).

21. The use of a means of detecting the presence or absence of mutation in the delta-catenin gene promoter or 5′ untranslated region in a biological sample from a subject for carrying out a method of claim 1.

22. A kit comprising a means of detecting the presence or absence of mutation in the delta-catenin gene promoter or 5′ untranslated region in a biological sample from a subject for carrying out a method of claim 1, said kit optionally including instructions for carrying out a method of claim 1.
